(19) United States
(12) Patent Application Publication
(10) Pub. No.: US 2008/0268064 A1
(43) Pub. Date: Oct. 30, 2008

(54) METHOD FOR TREATING CARTILAGE DEFECTS

(75) Inventor: Jennifer E. Woodell-May, Warsaw, IN (US)

Correspondence Address: HARNESS, DICKEY & PIERCE, P.L.C., P.O. BOX 828, BLOOMFIELD HILLS, MI, 48303, US

(73) Assignee: Biomet Biologics, Inc., Warsaw, IN (US)

(21) Appl. No.: 11/740,035

(22) Filed: Apr. 25, 2007

(51) Int. Cl. A61K 35/14; A61K 38/36; A61P 19/02

(52) U.S. Cl. 424/529; 128/898; 514/2

(57) ABSTRACT

Methods for treating a cartilage defect comprising fractioning blood to produce a blood component, shaping the cartilage defect to expose subchondral bone, microfracturing the subchondral bone, and applying the blood component to the microfractured subchondral bone. The blood component can comprise platelet-poor plasma.

### INTRODUCTION

The present technology relates to methods for treating cartilage defects to promote or enhance cartilage growth and repair.

There are a number of complex physiological steps and processes involved in tissue repair following damage to cartilage tissue caused by trauma, disease (such as osteoarthritis and osteochondrosis dissecans), excessive use (such as sports injuries), other disruption to the cartilage, or a lifetime of use. Influencing these processes and maximizing the strength of the repaired cartilage have been important in current medical research. Methods to enhance cartilage repair in terms of ease of use, healing rate, pain reduction, and efficacy are desirable.

### SUMMARY

The present technology provides methods for treating a cartilage defect comprising obtaining blood compatible with the subject, fractionating the blood to produce a blood component, shaping the cartilage defect to expose subchondral bone, microfracturing the subchondral bone to induce bleeding, and applying the blood component to the site of the microfractured subchondral bone. The blood component may be platelet-rich plasma. Methods include those wherein a clot is formed at the site of the subchondral bone after applying the blood component, and reinforcing the clot by mechanical or chemical techniques.

The blood component can act with blood and blood material released from the microfracture to effectively treat the cartilage defect, and stimulate production of hyaline cartilage. In this regard, administration of the blood component to the microfractured area can result in reduced pain, enhanced healing of the cartilage defect, and/or more complete healing of the cartilage defect compared to treatments using the blood component or microfracture alone.

Further areas of applicability will become apparent from the description provided herein. It should be understood that the description and specific examples are intended for purposes of illustration only and are not intended to limit the scope of the present technology.

### BRIEF DESCRIPTION OF THE DRAWINGS

The present technology will become more fully understood from the detailed description and the accompanying drawings, wherein:

FIG. 1 illustrates a representative cartilage defect on a subject in need of treatment according to one embodiment of the present technology;

FIG. 2 illustrates a shaped cartilage defect according to one embodiment of the present technology;

FIG. 3A and 3B illustrate various shaped cartilage defects after microfracture according to embodiments of the present technology;

FIG. 4 is a diagrammatic illustration of a representative method for treating a cartilage defect according to one embodiment of the present technology;

FIG. 5 is a front view of a representative device used for isolating a blood component according to one embodiment of the present technology;

FIG. 6 illustrates a microfractured cartilage defect filled with blood component according to one embodiment of the present technology; and

FIG. 7 illustrates a repaired cartilage defect having a membrane cover according to one embodiment of the present technology.

### DESCRIPTION

The following description of technology is merely exemplary in nature of the subject matter, manufacture, and use of one or more inventions, and is not intended to limit the scope, application, or uses of any specific invention claimed in this application or in such other applications as may be filed claiming priority to this application, or patents issuing therefrom.

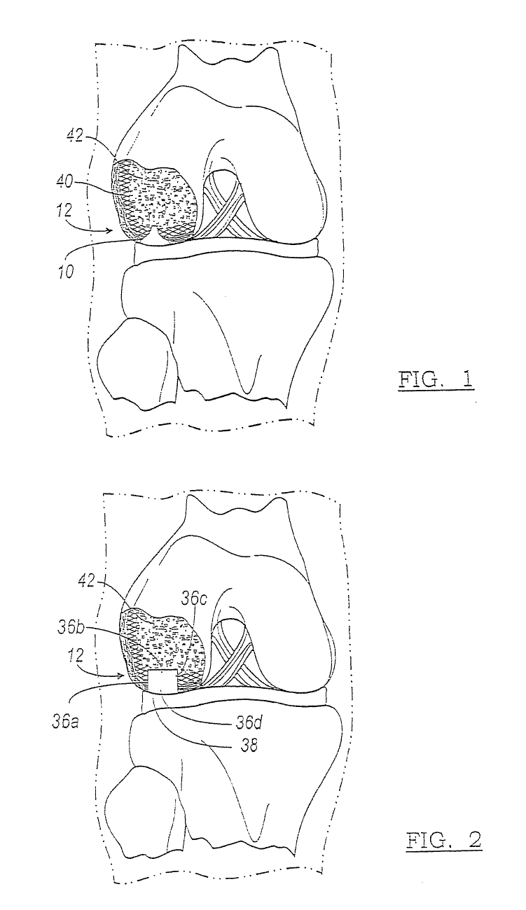

FIG. 1 illustrates a cartilage defect 10 in the knee 12 of a human subject. The cartilage defect 10 can be in the form of a focal cartilage defect or a cartilage abrasion, as non-limiting examples. Exemplary defect sites include, but are not limited to, a patella, a femoral condyle, a femoral head, an acetabulum, or any other articulating joint surfaces. It should be understood, however, that the cartilage defect may be any condition in which cartilage is inadequate for physiological or cosmetic purposes. In this regard, the cartilage defect 10 may include congenital cartilage defects, cartilage defects that result from or are symptomatic of disease (such as osteoarthritis and osteochondrosis dissecans), disorder, or trauma, natural aging, and those cartilage defects that are consequent to surgical or other medical procedures. The cartilage defect 10 may also be caused by excessive use (for example, sports injuries).

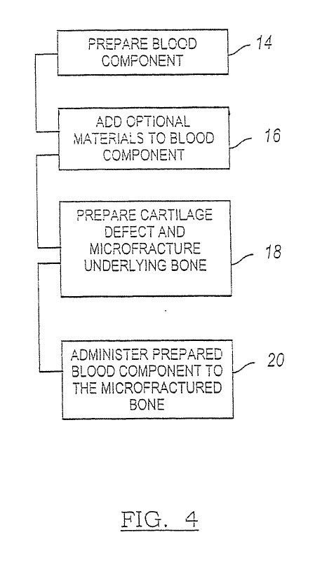

One embodiment for treating a cartilage defect 10 is shown diagrammatically in FIG. 4. In summary, a blood component is obtained at step 14. Optional components, such as one or more additives described below, may also be added to the blood component at step 16. The cartilage defect 10 is shaped in step 18, to expose underlying subchondral bone, which is then microfractured to induce bleeding from within the bone. The blood component (and any optional material) is then administered to the site of the microfractured cartilage defect 10 at step 20. Each of the aforementioned steps will be more fully discussed below.

As discussed above, a blood component is obtained at step 14. The blood component is preferably isolated from blood obtained from the subject exhibiting the cartilage defect 10 to be treated. The blood component may also be derived from bone marrow.

The blood component may comprise fractionated plasma in the form of platelet-rich plasma, platelet-poor plasma, or concentrated platelet-poor plasma. In this regard, the blood component comprising platelet-rich plasma may have an increased concentration of platelets relative to whole blood, and in some embodiments, the platelet concentration can be from about 3-fold to about 10-fold greater than the platelet concentration in whole blood.

The blood component can be obtained at step 14 by one or more methods, including filtration, cryoprecipitation, and density fractionation. Density fractionation techniques include single stage centrifugation, centrifugation in multiple stages, and continuous flow centrifugation.

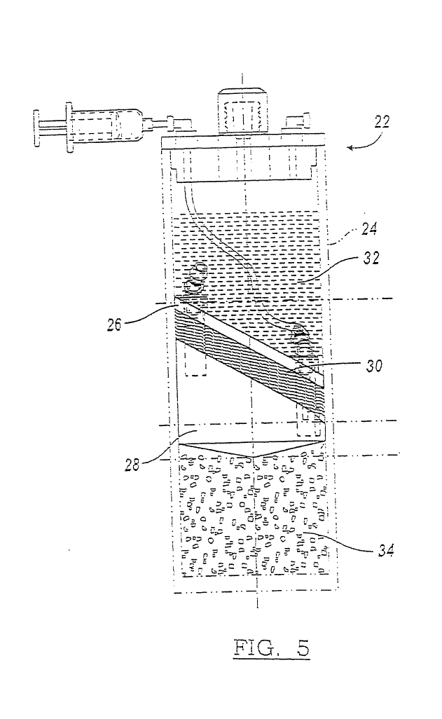

FIG. 5 illustrates a device that can be used for forming the blood component at step 14 by density fractionation. In this regard, the device 22 includes a container 24, such as a tube, that is placed in a centrifuge after being filled with blood. The container 24 includes a buoy system having an isolator 26 and a buoy 28. The buoy 28 has a selected density which is tuned to reach a selected equilibrium position upon centrifugation; this position lies between a more dense blood fraction and a less dense blood fraction. During centrifugation, the buoy 28 separates the blood within the container 24 into at least two fractions, without substantially commingling the fractions, by sedimenting to a position between the two fractions. In this regard, the isolator 26 and the buoy 28 define a layer comprising platelet-rich plasma 30, while less dense platelet-poor plasma 32 generally fractionates above the isolator 26, and more dense red blood cells 34 generally fractionate below the buoy 28. Following centrifugation, a syringe or tube may then be interconnected with a portion of the buoy system to extract one or more selected fractions for use as the blood component. Devices including those disclosed in FIG. 5 and associated methods are described in U.S. Patent Application Publication 2004/0251217, Leach et al., published Dec. 12, 2004; and U.S. Patent Application Publication 2005/0109716, Leach et al., published May 26, 2005; both of which are incorporated by reference herein. One such device that is commercially available is the GPS® Platelet Concentrate System, from Biomet Biologics, Inc. (Warsaw, Ind.).

Another example of a device that may be used in step 14 to isolate platelet-rich plasma or other blood components by density fractionation comprises a centrifugal drum separator and an erythrocyte capture trap. In one embodiment, the walls of the centrifugal drum separator are coated with a depth filter having pores and passageways that are sized to receive and entrap erythrocytes. Blood is placed in the centrifugal drum, and the drum is spun along its axis at sufficient speed so as to force erythrocytes from the blood into the depth filter. After spinning, the erythrocytes remain in the filter and the remaining platelet-rich plasma is extracted. The platelet-rich plasma may be concentrated by desiccation. Such devices include the Vortech™ Concentration System (Biomet Biologics, Inc., Warsaw, Ind.), and are disclosed in U.S. Patent Application Publication 2006/0175244, Dorian et al., published Aug. 10, 2006, and U.S. Patent Application Publication 2006/0175242, Dorian et al., published Aug. 10, 2006 which are hereby incorporated by reference.

Other devices that may be used for to obtain the blood component at step 14 are described, for example, in U.S. Pat. No. 6,398,972, Blasetti et al., issued Jun. 4, 2002; U.S. Pat. No. 6,649,072, Brandt et al., issued Nov. 18, 2003; U.S. Pat. 6,790,371, Dolecek, issued Sep. 14, 2004; U.S. Pat. No. 7,011,852, Sukavaneshvar et al., issued Mar. 14, 2006; U.S. Patent Application Publication 2005/0196874, Dorian et al., published Sep. 8, 2005; In addition to the GPS® Platelet Concentrate System, a variety of other commercially available devices may be used to obtain the blood component at step 14, including the Megellan™ Autologous Platelet Separator System, commercially available from Medtronic, Inc. (Minneapolis, Minn.); SmartPReP™, commercially available from Harvest Technologies Corporation (Plymouth, Mass.); DePuy (Warsaw, Ind.); the AutoloGel™ Process, commercially available from Cytomedix (Rockville, Md.), and the GenesisCS component concentrating system, available from EmCyte Corporation (Fort Myers, Fla.).

Referring again to FIG. 4, methods of the present technology optionally include the addition of one or more bioactive agents at step 16. Bioactive agents include platelet activators, stem cells and scaffold materials. The bioactive agents can be applied just prior to the administration of the blood component, concomitant with administration of the blood component, or following administration of the blood component to the cartilage defect 10 in step 20.

Platelet activators optionally included in step 16 may serve to activate one or more growth factors within platelets that may be in the blood material. Activation of the platelets by the platelet activators can be performed just prior to administration of the blood materials, concomitant with administration of the blood materials, or following administration of the blood materials to the cartilage defect in step 20. Platelet activators among those useful herein include thrombin, calcium chloride (CaCl₂), coagulation factors, and mixtures thereof. Coagulation factors include, but are not limited to, one or more of the following: V, VII, VIIa, IX, IXaβ, X, Xa, XI, XIa, XII, α-XIIa, β-XIIa, and XIII.

The bioactive agents added in step 16 may comprise stem cells, such as bone marrow-derived stem cells and adipose-derived stromal cells. Adipose-derived stromal cells may be obtained from processing of lipid tissue by standard liposuction and lipoaspiration methods known in the art. Adipose tissue may also be treated with digestive enzymes and with chelating agents that weaken the connections between neighboring cells, making it possible to disperse the tissue into a suspension of individual cells without appreciable cell breakage. Following disaggregation, the adipose stromal cells may be isolated from the suspension of cells and disaggregated tissue. A device such as the GPS® Platelet Concentrate System may be used to isolate adipose stromal cells.

A scaffold may be added in step 16 to contain, support, or retain the blood material at the cartilage defect site, or to facilitate migration of endogenous cells into the cartilage defect site. Scaffolds may be formed from porous or semi-porous, natural, synthetic or semisynthetic materials. Scaffold materials include those selected from the group consisting of bone (including cortical and cancellous bone), demineralized bone, ceramics, polymers, and combinations thereof. Suitable polymers may include collagen, including lyophilized or skin-derived collagen as disclosed in U.S. patent application Ser. No. 11/259,216 which is incorporated by reference herein. Polymers may also include gelatin, hyaluronic acid, chitosan, polyglycolic acid, polylactic acid, polypropylenefumarate, polyethylene glycol, and copolymers or combinations thereof. Ceramics include any of a variety of ceramic materials known in the art for use for implanting in bone, such as calcium phosphate (including tricalcium phosphate, tetracalcium phosphate, hydroxyapatite, and mixtures thereof). Concentrated platelet-poor plasma may also be used as a scaffold material, particularly in methods where a platelet-rich plasma blood component is percutaneously administered to the site of the cartilage defect 10 in step 20. Concentrated platelet-poor plasma may be prepared as described above in step 14.

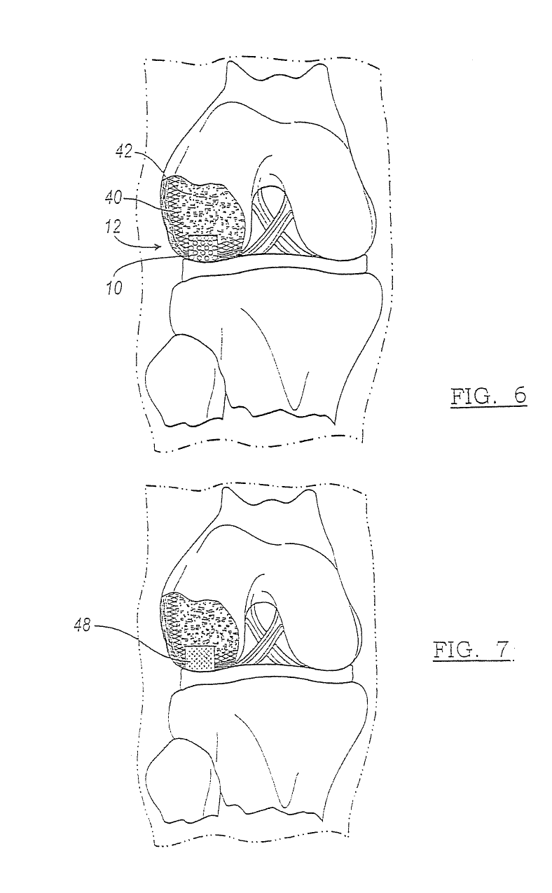

Referring to FIGS. 1-3B and 6, a microfracture procedure is performed at step 18 to prepare the cartilage defect. (Although step 18 is shown in FIG. 4 as following steps 14 and 16, the microfracture procedure of step 18 may be performed prior to or concurrent with steps 14 and 16.) Suitable microfracture procedures include those well known in the art. In general, a surgical site is first prepared either by an open surgical technique or arthroscopic surgical technique. Next, the cartilage defect 10 is shaped or prepared by removing an area of damaged cartilage to create a defined region 38 such as the region contained in walls 36a, 36b, 36c, 36d about the cartilage defect 10. The removal of the damaged cartilage can include resecting or contouring the cartilage to provide a region of healthy cartilage and/or underlying bone upon which to administer the blood component (and any additional material).

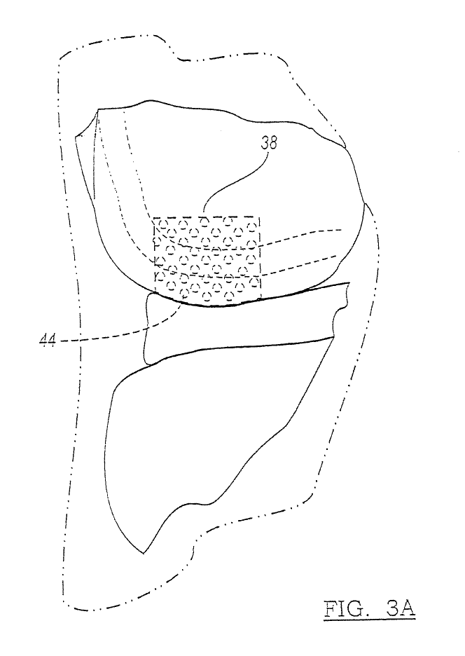

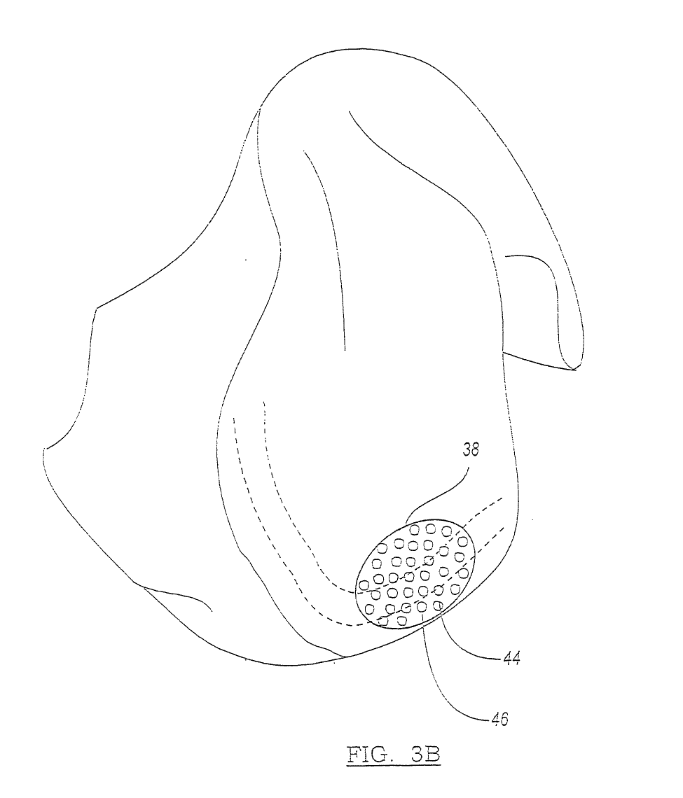

As shown in FIGS. 3A and 3B, the defined region 38 can be any suitable shape, for example, a squared shape or a circular shape. The amount of material to be removed and the contour of the area may dictate the shape of the defined region 38. Providing the healthy surrounding cartilage by shaping or contouring the defect site, maximizes the effectiveness of the microfracture therapy and facilitates integration of new healthy cartilage into the cartilage defect 10. In some embodiments, an underlying area of calcified cartilage is exposed.

After the overlying cartilage is removed and shaped, calcified cartilage 40 is removed from the defect site. The calcified cartilage 40 can be scrapped off with a scalpel, removed using an abrasive bit on a surgical drill, or any other suitable device or technique. When the calcified cartilage 40 is removed, the underlying subchondral bone 42 is exposed. An awl or pick is advanced into the subchondral bone 42 to create small holes or “microfractures” 44 in the bone as shown in FIGS. 3A and 3B.

The microfractures are sufficiently spaced apart to maintain the integrity of the subchondral bone 42. It is understood that the size of the space or “bony bridge” 46 between microfractures will vary based on the size of the cartilage defect, the health of the subchondral bone 42, and the load bearing properties of the particular cartilage defect 10. Typically, the space 46 is about 4 mm.

The microfracture procedure preferably induces bleeding and seeping of blood material from the subchondral bone 42 into the cartilage defect 10. This blood material may comprise whole blood and various blood components, including bone marrow and accompanying stem cells. The blood may seep into the microfracture 44 holes and at least partially fill the cartilage defect 10. The induced bleeding forms a blood clot that releases cartilage building cells from the bone marrow. It is beneficial to shape the surrounding cartilage such that the microfracture blood containing the cartilage building cells can integrate into a healthy surrounding tissue.

In various embodiments, an isolation device can be placed over the cartilage defect 10 to isolate the cartilage defect 10 from an ambient fluid or from a surrounding tissue. Suitable isolation devices are disclosed in U.S. patent application Ser. No. 11/739,768, filed Apr. 25, 2007, Stone, which is incorporated by reference. Ambient fluids include blood, for example, or extracorporeal fluids used to wash the defect site. The surrounding tissue may include soft tissues, such as muscle, connective tissues, fat, and the surrounding tissue can also include bony tissue. By isolating the cartilage defect 10, the surrounding tissue is pushed away from and contained outside of the cartilage defect 10. This is particularly useful for focal cartilage defects or with microfractures as the isolation prevents ambient tissue from lying over or in the cartilage defect, thereby blocking the delivery of the discrete and localized therapy. Moreover, this prevents the dilution of the blood component that is applied to the subchondral bone in step 20.

Referring again to FIG. 4, the blood component or therapeutic composition is administered to the cartilage defect at step 20. Administering the blood component or therapeutic composition can include any biomedically acceptable process or procedure by which the blood component or therapeutic composition is implanted, injected, sprayed, applied, filled or otherwise administered in, on, or in proximity to the site of the cartilage defect 10 so as to have a beneficial effect to the cartilage.

The blood component or therapeutic composition can be administered to the cartilage defect to at least partially fill the cartilage defect 10. In various embodiments, the blood component or therapeutic composition can be administered to only fill the microfracture holes 44. In another embodiment, the blood component or therapeutic composition can be administered such that the shaped and microfractured area is flush with the surrounding healthy cartilage. Providing a flush repair is advantageous where the cartilage defect is an articular cartilage defect. A flush repair may also help to minimize pain after the repair.

The blood component or therapeutic composition can be retained in the defect site by allowing a clot to form naturally therein using endogenous clotting agents, inducing clot formation at the defect site, by using a barrier layer, by applying an adhesive material, and combinations thereof. In one embodiment, the clot forms in the cartilage defect 10 when platelets from blood seeped from the subchondral bone 42 clot without the addition of extracorporeal clotting agents. In other embodiments, the formation of the clot can be expedited by the addition of a platelet activator, such as those listed above regarding step 18. The clot preferably covers and secures the defined region 38 of the cartilage defect 10 in a short amount of time. The quick clotting time minimizes the exposure of the subchondral bone 42 and minimizes exposure of the cartilage defect 10 having the blood resultant from the microfracture therein from being diluted by ambient fluids. In various embodiments, the clot is formed in less than 2 minutes, less than 1 minute, or less than 30 seconds. In other embodiments, the clot is formed in from about 2 to about 50 seconds, from about 10 to about 40 seconds, from about 20 to about 30 seconds, or from about 10 to about 25 seconds.

As shown in FIG. 7, a barrier layer 48 may be used to secure the microfractured defect site 10. Exemplary barrier layers include hydrogel, chitosan, or other scaffolds, such as discussed above in step 18, as well as concentrated platelet-poor plasma, a periosteal flap, a membrane, and combinations thereof. The barrier layer 48 is placed over the cartilage defect 10 and is secured to the surrounding healthy cartilage. An adhesive material such as fibrin glue, bioadhesives, sealants, and combinations thereof, can also be used to secure the blood component in the cartilage defect 10. Combinations of the above can also be used to secure the clot in the cartilage defect. For example, a periosteal flap can be secured to the surrounding healthy cartilage by covering the periosteal flap with a fibrin glue. In one method, platelet-poor plasma is administered followed by percutaneous administration of concentrated platelet-poor plasma to substantially cover the site of the cartilage defect 20.

The examples and other embodiments described herein are exemplary and not intended to be limiting in describing the full scope of compositions and methods of this technology. Equivalent changes, modifications and variations of specific embodiments, materials, compositions and methods may be made within the scope of the present technology, with substantially similar results.

1. A method for treating a cartilage defect in a human subject comprising:
obtaining blood compatible with the subject;
fractioning the blood to produce a blood component, said blood component selected from the group consisting of platelet-poor plasma, concentrated platelet-poor plasma, platelet-rich plasma, and combinations thereof;
shaping the cartilage defect to expose subchondral bone;
microfracturing the subchondral bone; and
applying the blood component to the site of the microfractured subchondral bone to substantially fill the cartilage defect with the blood component.

2. The method of claim 1, wherein the blood component comprises platelet-rich plasma.

3. The method of claim 1, further comprising resecting a portion of a surrounding cartilage at the cartilage defect.

4. (canceled)

5. The method of claim 1, wherein the applying comprises delivering a sufficient amount of the blood component to provide a cartilage fill area that is substantially flush with surrounding healthy cartilage.

6. The method of claim 1, wherein shaping the cartilage defect to expose an underlying bone comprises removing a layer of calcified cartilage at the cartilage defect.

7. The method of claim 2, further comprising administering to the cartilage defect a platelet activator selected from the group consisting of thrombin, CaCl₂, a coagulation factor, and combinations thereof.

8. The method of claim 7, further comprising simultaneously delivering the blood component and a platelet activator to the cartilage defect.

9. The method of claim 8, wherein a clot is formed in less than 30 seconds.

10. The method of claim 2, further comprising administering concentrated platelet-poor plasma to the cartilage defect.
